(19) United States
(12) Patent Application Publication
(10) Pub. No.: US 2022/0313088 A1
(43) Pub. Date: Oct. 6, 2022

(54) SYSTEMS AND METHODS FOR MOTION DETECTION

(71) Applicant: SHANGHAI UNITED IMAGING HEALTHCARE CO., LTD., Shanghai (CN)

(72) Inventors: Xinyuan XIA, Shanghai (CN); Yiran LI, Houston, TX (US); Lingzhi HU, Houston, TX (US)

(73) Assignee: SHANGHAI UNITED IMAGING HEALTHCARE CO., LTD., Shanghai (CN)

(21) Appl. No.: 17/647,173

(22) Filed: Jan. 6, 2022

(30) Foreign Application Priority Data
Apr. 2, 2021 (CN) 202110359361.5

(51) Int. Cl. A61B 5/00

(52) U.S. Cl. CPC A61B 5/0013; A61B 5/0044

(57) ABSTRACT

The present disclosure is related to systems and methods for motion detection. The method includes obtaining, via a first detection device, original cardiac motion data of a subject located in a field of view (FOV) of a medical device. The method includes obtaining, via a second detection device, detection data of the subject. The detection data includes at least one of posture data of the subject or physiological motion data of the subject. The method includes determining target cardiac motion data of the subject by correcting, based on the detection data, the original cardiac motion data.

### CROSS-REFERENCE TO RELATED APPLICATIONS

This application claims priority of Chinese Patent Application No. 202110359361.5, filed on Apr. 2, 2021, and the contents of which are hereby incorporated by reference.

### TECHNICAL FIELD

This disclosure generally relates to systems and methods for medical imaging, and more particularly, relates to systems and methods for motion detection in medical imaging.

### BACKGROUND

Medical systems, such as CT scanners, MRI scanners, PET scanners, are widely used for creating images of interior of a patient's body for, e.g., medical diagnosis and/or treatment purposes. A motion (e.g., a posture motion, a physiological motion) of the subject during a scan may affect imaging quality by causing, for example, motion artifacts in a resulting image, which in turn may hinder an accurate detection, localization, and/or quantification of possible lesions (e.g., a tumor). Therefore, it is desirable to provide effective systems or methods for motion detection in the medical imaging.

### SUMMARY

According to an aspect of the present disclosure, a method may be implemented on a computing device having one or more processors and one or more storage devices. The method may include obtaining, via a first detection device, original cardiac motion data of a subject located in a field of view (FOV) of a medical device. The method may include obtaining, via a second detection device, detection data of the subject. The detection data may include at least one of posture data of the subject or physiological motion data of the subject. The method may include determining target cardiac motion data of the subject by correcting, based on the detection data, the original cardiac motion data.

In some embodiments, the method may include generating, based on the target cardiac motion data, a control signal for controlling the medical device to scan the subject. The method may include causing the medical device to perform, according to the control signal, a scan on the subject.

In some embodiments, the physiological motion data may include respiratory motion data. The method may include obtaining correction data by extracting at least one of the posture data or the respiratory motion data from the detection data. The method may include determining the target cardiac motion data of the subject by correcting, based on the correction data, the original cardiac motion data.

In some embodiments, the method may include determining the target cardiac motion data of the subject by removing the correction data from the original cardiac motion data.

In some embodiments, the correction data may include respiratory motion data. The method may include obtaining transformed cardiac motion data by transforming the original cardiac motion data from a time domain to a frequency domain. The method may include obtaining transformed respiratory motion data by transforming the respiratory motion data from the time domain to the frequency domain. The method may include obtaining candidate cardiac motion data by subtracting the transformed respiratory motion data from the transformed cardiac motion data. The method may include determining the target cardiac motion data by transforming the candidate cardiac motion from the frequency domain to the time domain.

In some embodiments, the control signal may involve a gating technique according to which the medical device performs the scan.

In some embodiments, the first detection device may include at least one of a first radar sensor, an electrocardiographic device, or a pulse measuring device.

In some embodiments, the second detection device may include at least one of a second radar sensor, an image acquisition device, a pressure sensor, or an acceleration sensor.

In some embodiments, a first emission frequency of the first radar sensor may be lower than a second emission frequency of the second radar sensor.

In some embodiments, the first radar sensor may be a Doppler radar. The first emission frequency may be in a range of 600 MHz˜2.4 GHz. The second radar sensor may be a millimeter wave radar sensor.

In some embodiments, a plurality of second radar sensors may be mounted on different portions of a scanning cavity of the medical device to monitor the subject from different directions.

In some embodiments, the medical device may include at least one of a magnetic resonance imaging (MRI) device, a positron emission tomography (PET) device, a single-photon emission computed tomography (SPECT) device, a computed tomography (CT) device, or an X-ray imaging device.

According to another aspect of the present disclosure, a system may include at least one storage device storing a set of instructions, and at least one processor in communication with the at least one storage device. When executing the stored set of instructions, the at least one processor may cause the system to perform a method. The method may include obtaining, via a first detection device, original cardiac motion data of a subject located in a field of view (FOV) of a medical device. The method may include obtaining, via a second detection device, detection data of the subject. The detection data may include at least one of posture data of the subject or physiological motion data of the subject. The method may include determining target cardiac motion data of the subject by correcting, based on the detection data, the original cardiac motion data.

According to another aspect of the present disclosure, a non-transitory computer readable medium may include at least one set of instructions. When executed by at least one processor of a computing device, the at least one set of instructions may cause the at least one processor to effectuate a method. The method may include obtaining, via a first detection device, original cardiac motion data of a subject located in a field of view (FOV) of a medical device. The method may include obtaining, via a second detection device, detection data of the subject. The detection data may include at least one of posture data of the subject or physiological motion data of the subject. The method may include determining target cardiac motion data of the subject by correcting, based on the detection data, the original cardiac motion data.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating exemplary hardware and/or software components of an exemplary computing device on which the processing device 120 may be implemented according to some embodiments of the present disclosure;

FIG. 3 is a schematic diagram illustrating exemplary hardware and/or software components of an exemplary mobile device according to some embodiments of the present disclosure;

FIG. 4 is a schematic diagram illustrating an exemplary processing device according to some embodiments of the present disclosure;

FIG. 5 is a flowchart illustrating an exemplary process for determining target cardiac motion data of a subject according to some embodiments of the present disclosure;

FIGS. 6 and 7 are schematic diagrams illustrating an exemplary medical system according to some embodiments of the present disclosure; and

FIG. 8 is a flowchart illustrating an exemplary process for determining target cardiac motion data of a subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Also, the term “exemplary” is intended to refer to an example or illustration.

It will be understood that the terms “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure.

Spatial and functional relationships between elements are described using various terms, including “connected,” “attached,” and “mounted.” Unless explicitly described as being “direct,” when a relationship between first and second elements is described in the present disclosure, that relationship includes a direct relationship where no other intervening elements are present between the first and second elements, and also an indirect relationship where one or more intervening elements are present (either spatially or functionally) between the first and second elements. In contrast, when an element is referred to as being “directly” connected, attached, or positioned to another element, there are no intervening elements present. Other words used to describe the relationship between elements should be interpreted in a like fashion (e.g., “between,” versus “directly between,” “adjacent,” versus “directly adjacent,” etc.).

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The term “image” in the present disclosure is used to collectively refer to image data (e.g., scan data, projection data) and/or images of various forms, including a two-dimensional (2D) image, a three-dimensional (3D) image, a four-dimensional (4D), etc. The term “anatomical structure” in the present disclosure may refer to gas (e.g., air), liquid (e.g., water), solid (e.g., stone), cell, tissue, organ of a subject, or any combination thereof, which may be displayed in an image and really exist in or on the subject's body. The term “region,” “location,” and “area” in the present disclosure may refer to a location of an anatomical structure shown in the image or an actual location of the anatomical structure existing in or on the subject's body, since the image may indicate the actual location of a certain anatomical structure existing in or on the subject's body. The term “an image of a subject” may be referred to as the subject for brevity.

An aspect of the present disclosure relates to a system and method for motion detection in a medical procedure. According to some embodiments of the present disclosure, a processing device may obtain, via a first detection device (e.g., a first radar sensor), original cardiac motion data of a subject located in a field of view (FOV) of a medical device. As used herein, an FOV of a medical device refers to an area or region scanned by the medical device during a scan of a subject. The medical device may include an imaging device, a treatment device, or a combination thereof. A scan of a subject by a medical device may include an imaging scan or a treatment of the subject using the medical device. The processing device may obtain, via a second detection device (e.g., a second radar sensor), detection data of the subject. The detection data may include at least one of posture data of the subject or physiological motion data of the subject. The processing device may determine target cardiac motion data of the subject by correcting, based on the detection data, the original cardiac motion data.

In some embodiments, the first detection device may be a first radar sensor (e.g., a Doppler radar). The second detection device may be a second radar sensor (e.g., a millimeter wave radar sensor). A first emission frequency of the first radar sensor may be lower than a second emission frequency of the second radar sensor. In some embodiments, at least one of the first radar sensor or the second radar sensor may be a non-contact detection device. As used herein, a non-contact detection device indicates that the detection device does not need to be in physical contact with a subject when detecting data relating to a motion of the subject or that the detection of data relating to the motion of the subject by the detection device does not depend on the detection device being in contact with the subject. As used herein, A being in physical contact with B indicates that A contacts B and is not separated from B by an item (e.g., a solid item, a layer of a fluid (e.g., a liquid, air, etc.) of any shape (e.g., a thin layer, a stripe, etc.). In some embodiments, the first radar sensor may be placed above the chest of the subject by a certain distance. The second radar sensor may be mounted outside the FOV of the medical device. For example, the medical device may be a magnetic resonance imaging (MRI) device, and the second radar sensor may be mounted on a radio frequency (RF) coil of the MRI device.

Accordingly, by using the first radar sensor with a relatively low emission frequency to monitor a motion of a relatively small area of the subject (e.g., an area of the subject that is in a vicinity of the heart of the subject), the original cardiac motion data of the subject may be obtained accurately. By using the second radar sensor with a relatively high emission frequency to monitor a motion of a relatively large area of the subject (e.g., a chest and abdomen area of the subject, a whole body area of the subject), the detection data (e.g., respiratory motion data, posture data) of the subject may be obtained accurately. In addition, the signal interference between the second radar sensor and the medical device may be reduced or eliminated by mounting the second radar sensor outside the FOV of the medical device. Furthermore, in a conventional way, during a scan of a subject, one or more electrodes may be attached to the body of the subject in order to detect the physiological motion of the subject, which may cause discomfort to the subject and/or interfere with the imaging of the subject. Compared to a contact detection device (in which the detection device needs to be in physical contact with a subject for detecting motion data of the subject), the non-contact detection device disclosed herein may reduce the discomfort of the subject, reduce interference of the imaging by a contact detection device attached on the subject, and/or avoid the procedure and time needed for setting up such a contact detection device on the subject, which in turn may reduce the setup time of the medical device. Moreover, a control signal for controlling the medical device to scan the subject may be generated based on target motion data (e.g., target cardiac motion data), which may reduce physiological motion-induced artifacts in a resulting image.

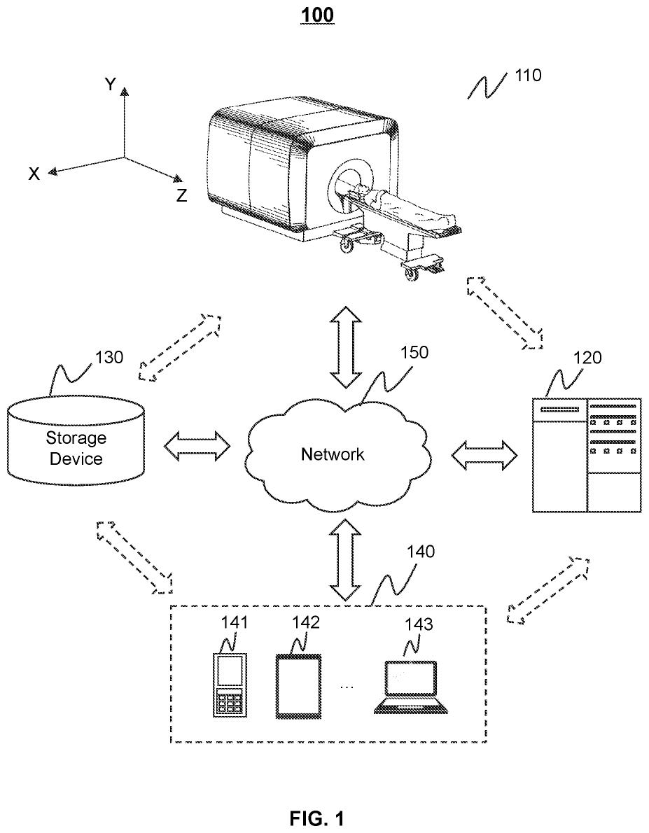

FIG. 1 is a schematic diagram illustrating an exemplary medical system according to some embodiments of the present disclosure. As illustrated, a medical system 100 may include a medical device 110, a processing device 120, a storage device 130, a terminal 140, and a network 150. The components of the medical system 100 may be connected in one or more of various ways. Merely by way of example, as illustrated in FIG. 1, the medical device 110 may be connected to the processing device 120 directly as indicated by the bi-directional arrow in dotted lines linking the medical device 110 and the processing device 120, or through the network 150. As another example, the storage device 130 may be connected to the medical device 110 directly as indicated by the bi-directional arrow in dotted lines linking the medical device 110 and the storage device 130, or through the network 150. As still another example, the terminal 140 may be connected to the processing device 120 directly as indicated by the bi-directional arrow in dotted lines linking the terminal 140 and the processing device 120, or through the network 150.

The medical device 110 may be configured to acquire imaging data relating to a subject. The imaging data relating to a subject may include an image (e.g., an image slice), projection data, or a combination thereof. In some embodiments, the imaging data may be a two-dimensional (2D) imaging data, a three-dimensional (3D) imaging data, a four-dimensional (4D) imaging data, or the like, or any combination thereof. The subject may be biological or non-biological. For example, the subject may include a patient, a man-made object, etc. As another example, the subject may include a specific portion, an organ, and/or tissue of the patient. Specifically, the subject may include the head, the neck, the thorax, the heart, the stomach, a blood vessel, soft tissue, a tumor, or the like, or any combination thereof. In the present disclosure, “object” and “subject” are used interchangeably.

In some embodiments, the medical device 110 may include a single modality imaging device. For example, the medical device 110 may include a positron emission tomography (PET) device, a single-photon emission computed tomography (SPECT) device, a magnetic resonance imaging (MRI) device (also referred to as an MR device, an MR scanner), a computed tomography (CT) device, an ultrasound (US) device, an X-ray imaging device, or the like, or any combination thereof. In some embodiments, the medical device 110 may include a multi-modality imaging device. Exemplary multi-modality imaging devices may include a PET-CT device, a PET-MRI device, a SPET-CT device, or the like, or any combination thereof. The multi-modality imaging device may perform multi-modality imaging simultaneously. For example, the PET-CT device may generate structural X-ray CT data and functional PET data simultaneously in a single scan. The PET-MRI device may generate MRI data and PET data simultaneously in a single scan.

In the present disclosure, the X axis, the Y axis, and the Z axis shown in FIG. 1 may form an orthogonal coordinate system. The X axis and the Z axis shown in FIG. 1 may be horizontal, and the Y axis may be vertical. As illustrated, the positive X direction along the X axis may be from the right side to the left side of the medical device 110 seen from the direction facing the front of the medical device 110; the positive Y direction along the Y axis shown in FIG. 1 may be from the lower part to the upper part of the medical device 110; the positive Z direction along the Z axis shown in FIG. 1 may refer to a direction in which the subject is moved out of a scanning channel (or referred to as a bore) of the medical device 110.

Merely by way of example, the medical device may be an MRI device. The MRI device may scan a subject located within its FOV and generate MR image data relating to the subject. The MR image data may include k-space data, MR signals, an MR image, etc. The MR image data may be acquired by the MRI device via scanning the subject using a pulse sequence. Exemplary pulse sequences may include a spin-echo sequence, a gradient echo sequence, a diffusion sequence, an inversion recovery sequence, or the like, or any combination thereof. For example, the spin-echo sequence may include a fast spin-echo (FSE), a turbo spin-echo (TSE), a rapid acquisition with relaxation enhancement (RARE), a half-Fourier acquisition single-shot turbo spin-echo (HASTE), a turbo gradient spin echo (TGSE), or the like, or a combination thereof.

The processing device 120 may process data and/or information obtained from the medical device 110, the storage device 130, and/or the terminal(s) 140. For example, the processing device 120 may obtain, via a first detection device, original cardiac motion data of a subject located in an FOV of a medical device. As another example, the processing device 120 may obtain, via a second detection device, detection data of the subject. As another example, the processing device 120 may determine target cardiac motion data of the subject by correcting, based on the detection data, the original cardiac motion data. In some embodiments, the processing device 120 may be a single server or a server group. The server group may be centralized or distributed. In some embodiments, the processing device 120 may be local or remote. For example, the processing device 120 may access information and/or data from the medical device 110, the storage device 130, and/or the terminal(s) 140 via the network 150. As another example, the processing device 120 may be directly connected to the medical device 110, the terminal(s) 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the terminal 140. In some embodiments, the processing device 120 may be part of the medical device 110.

The storage device 130 may store data, instructions, and/or any other information. In some embodiments, the storage device 130 may store data obtained from the medical device 110, the processing device 120, and/or the terminal(s) 140. The data may include image data acquired by the processing device 120, algorithms and/or models for processing the image data, etc. For example, the storage device 130 may store original cardiac motion data of a subject obtained from one or more detection devices. As another example, the storage device 130 may store detection data of a subject obtained from one or more detection devices. As another example, the storage device 130 may store target cardiac motion data of a subject determined by the processing device 120. In some embodiments, the storage device 130 may store data and/or instructions that the processing device 120 and/or the terminal 140 may execute or use to perform exemplary methods described in the present disclosure. In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memories may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), a high-speed RAM, etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more other components in the medical system 100 (e.g., the processing device 120, the terminal(s) 140). One or more components in the medical system 100 may access the data or instructions stored in the storage device 130 via the network 150. In some embodiments, the storage device 130 may be integrated into the medical device 110.

The terminal(s) 140 may be connected to and/or communicate with the medical device 110, the processing device 120, and/or the storage device 130. In some embodiments, the terminal 140 may include a mobile device 141, a tablet computer 142, a laptop computer 143, or the like, or any combination thereof. For example, the mobile device 141 may include a mobile phone, a personal digital assistant (PDA), a gaming device, a navigation device, a point of sale (POS) device, a laptop, a tablet computer, a desktop, or the like, or any combination thereof. In some embodiments, the terminal 140 may include an input device, an output device, etc. The input device may include alphanumeric and other keys that may be input via a keyboard, a touchscreen (for example, with haptics or tactile feedback), a speech input, an eye tracking input, a brain monitoring system, or any other comparable input mechanism. Other types of the input device may include a cursor control device, such as a mouse, a trackball, or cursor direction keys, etc. The output device may include a display, a printer, or the like, or any combination thereof.

The network 150 may include any suitable network that can facilitate the exchange of information and/or data for the medical system 100. In some embodiments, one or more components of the medical system 100 (e.g., the medical device 110, the processing device 120, the storage device 130, the terminal(s) 140, etc.) may communicate information and/or data with one or more other components of the medical system 100 via the network 150. For example, the processing device 120 and/or the terminal 140 may obtain an image from the medical device 110 via the network 150. As another example, the processing device 120 and/or the terminal 140 may obtain information stored in the storage device 130 via the network 150. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (VPN), a satellite network, a telephone network, routers, hubs, witches, server computers, and/or any combination thereof. For example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the medical system 100 may be connected to the network 150 to exchange data and/or information.

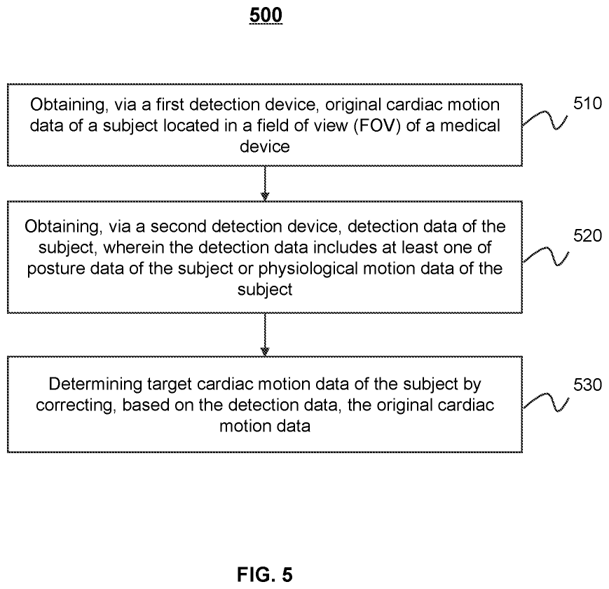

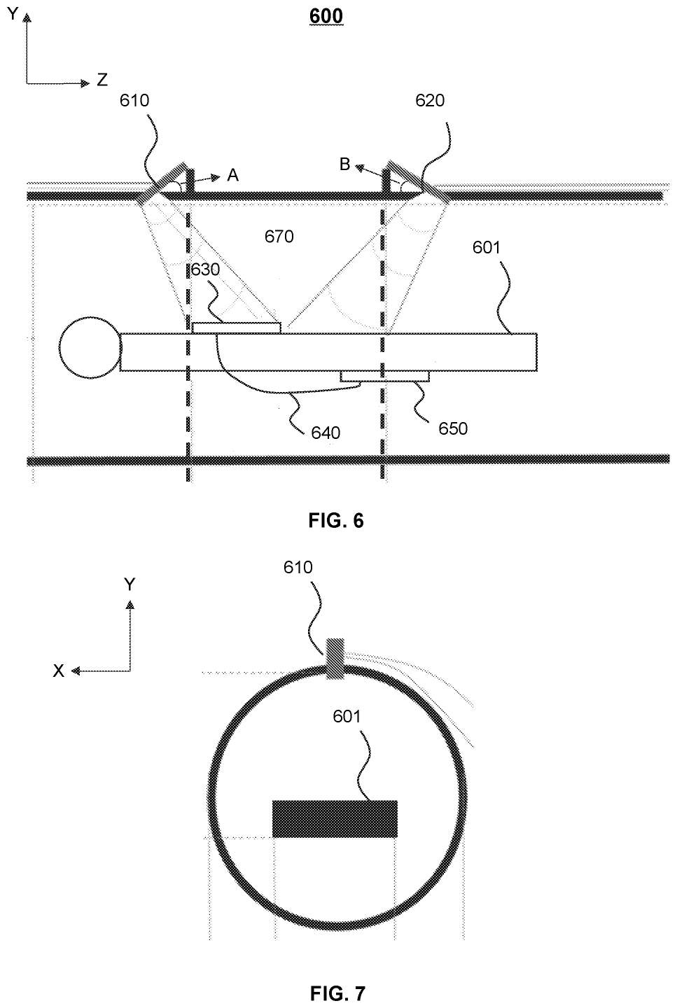

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. However, those variations and modifications do not depart the scope of the present disclosure. In some embodiments, the medical system 100 may include a first detection device and a second detection device (e.g., a second radar sensor 610, a second radar sensor 620 as illustrated in FIG. 6). The first detection device may be configured to obtain original cardiac motion data of a subject before and/or during a scan (e.g., an MR scan) of the subject. The second detection device may be configured to obtain detection data (e.g., posture data, physiological motion data) of a subject before and/or during a scan (e.g., an MR scan) of the subject. In some embodiments, at least one of the first detection device or the second detection device may be non-contact detection devices. For example, the first detection device may be a first radar sensor (e.g., a Doppler radar). The second detection device may be a second radar sensor (e.g., a millimeter wave radar sensor). More descriptions of the first detection device and the second detection device may be found elsewhere in the present disclosure (e.g., FIGS. 5-8 and descriptions thereof).

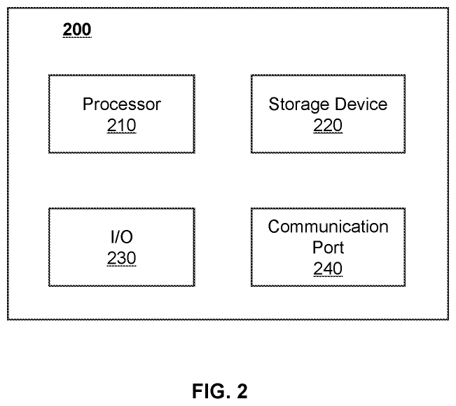

FIG. 2 is a schematic diagram illustrating exemplary hardware and/or software components of an exemplary computing device on which the processing device 120 may be implemented according to some embodiments of the present disclosure. As illustrated in FIG. 2, a computing device 200 may include a processor 210, a storage device 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein. For example, the processor 210 may process image data obtained from the medical device 110, the terminal device 140, the storage device 130, and/or any other component of the medical system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combination thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage device 220 may store data/information obtained from the medical device 110, the terminal device 140, the storage device 130, and/or any other component of the medical system 100. The storage device 220 may be similar to the storage device 130 described in connection with FIG. 1, and the detailed descriptions are not repeated here.

The I/O 230 may input and/or output signals, data, information, etc. In some embodiments, the I/O 230 may enable a user interaction with the processing device 120. In some embodiments, the I/O 230 may include an input device and an output device. Examples of the input device may include a keyboard, a mouse, a touchscreen, a microphone, a sound recording device, or the like, or a combination thereof. Examples of the output device may include a display device, a loudspeaker, a printer, a projector, or the like, or a combination thereof. Examples of the display device may include a liquid crystal display (LCD), a light-emitting diode (LED)-based display, a flat panel display, a curved screen, a television device, a cathode ray tube (CRT), a touchscreen, or the like, or a combination thereof.

The communication port 240 may be connected to a network (e.g., the network 150) to facilitate data communications. The communication port 240 may establish connections between the processing device 120 and the medical device 110, the terminal device 140, and/or the storage device 130. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. In some embodiments, the communication port 240 may be and/or include a standardized communication port, such as RS232, RS485. In some embodiments, the communication port 240 may be a specially designed communication port. For example, the communication port 240 may be designed in accordance with the digital imaging and communications in medicine (DICOM) protocol.

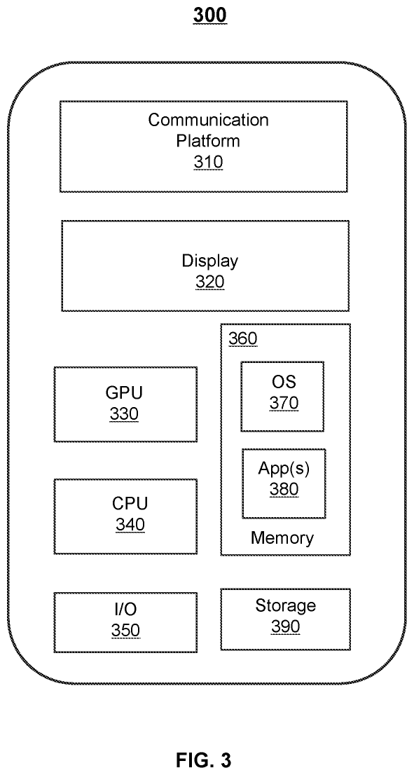

FIG. 3 is a schematic diagram illustrating exemplary hardware and/or software components of an exemplary mobile device according to some embodiments of the present disclosure. In some embodiments, the terminal device 140 and/or the processing device 120 may be implemented on a mobile device 300, respectively.

As illustrated in FIG. 3, the mobile device 300 may include a communication platform 310, a display 320, a graphics processing unit (GPU) 330, a central processing unit (CPU) 340, an I/O 350, a memory 360, and storage 390. In some embodiments, any other suitable component, including but not limited to a system bus or a controller (not shown), may also be included in the mobile device 300.

In some embodiments, the communication platform 310 may be configured to establish a connection between the mobile device 300 and other components of the medical system 100, and enable data and/or signal to be transmitted between the mobile device 300 and other components of the medical system 100. For example, the communication platform 310 may establish a wireless connection between the mobile device 300 and the medical device 110, and/or the processing device 120. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. The communication platform 310 may also enable the data and/or signal between the mobile device 300 and other components of the medical system 100. For example, the communication platform 310 may transmit data and/or signals inputted by a user to other components of the medical system 100. The inputted data and/or signals may include a user instruction. As another example, the communication platform 310 may receive data and/or signals transmitted from the processing device 120. The received data and/or signals may include imaging data acquired by the medical device 110.

In some embodiments, a mobile operating system (OS) 370 (e.g., iOS™ Android™, Windows Phone™, etc.) and one or more applications (App(s)) 380 may be loaded into the memory 360 from the storage 390 in order to be executed by the CPU 340. The applications 380 may include a browser or any other suitable mobile apps for receiving and rendering information from the processing device 120. User interactions with the information stream may be achieved via the I/O 350 and provided to the processing device 120 and/or other components of the medical system 100 via the network 150.

To implement various modules, units, and their functionalities described in the present disclosure, computer hardware platforms may be used as the hardware platform(s) for one or more of the elements described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result the drawings should be self-explanatory.

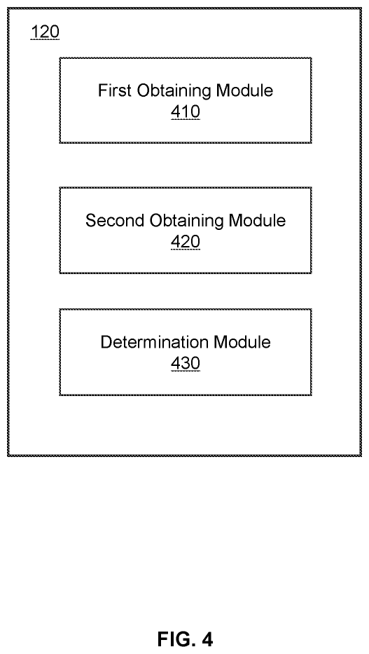

FIG. 4 is a schematic diagram illustrating an exemplary processing device according to some embodiments of the present disclosure. In some embodiments, the processing device 120 may include a first obtaining module 410, a second obtaining module 420, and a determination module 430.

The first obtaining module 410 may be configured to obtain data and/or information associated with the medical system 100. The data and/or information associated with the medical system 100 may include first detection information, original cardiac motion data, or the like, or any combination thereof. For example, the first obtaining module 410 may obtain, via a first detection device, original cardiac motion data of a subject located in an FOV of a medical device (e.g., the medical device 110). More descriptions for obtaining the original cardiac motion data may be found elsewhere in the present disclosure (e.g., operation 510 in FIG. 5, and descriptions thereof). In some embodiments, the first obtaining module 410 may obtain the data and/or the information associated with the medical system 100 from one or more components (e.g., the medical device 110, the storage device 130, the terminal 140, the first detection device) of the medical system 100 via the network 150.

The second obtaining module 420 may be configured to obtain data and/or information associated with the medical system 100. The data and/or information associated with the medical system 100 may include second detection information, detection data, or the like, or any combination thereof. For example, the second obtaining module 420 may obtain, via a second detection device, detection data of the subject. More descriptions for obtaining the detection data may be found elsewhere in the present disclosure (e.g., operation 520 in FIG. 5, and descriptions thereof). In some embodiments, the second obtaining module 420 may obtain the data and/or the information associated with the medical system 100 from one or more components (e.g., the medical device 110, the storage device 130, the terminal 140, the second detection device) of the medical system 100 via the network 150.

The determination module 430 may be configured to determine data and/or information associated with the medical system 100. For example, the determination module 430 may determine target cardiac motion data of a subject by correcting, based on detection data, original cardiac motion data. More descriptions for determining the target cardiac motion data may be found elsewhere in the present disclosure (e.g., operation 530 in FIG. 5, and descriptions thereof). As another example, the determination module 430 may generate, based on target cardiac motion data, a control signal for controlling a medical device to scan a subject. More descriptions for generating the control signal may be found elsewhere in the present disclosure (e.g., operation 540 in FIG. 5, and descriptions thereof).

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be combined into a single module. For example, the first obtaining module 410 and the second obtaining module 420 may be combined into a single module. In some embodiments, one or more modules may be added or omitted in the processing device 120. For example, the processing device 120 may further include a storage module (not shown in FIG. 4) configured to store data and/or information (e.g., original cardiac motion data, the detection data, the target cardiac motion data) associated with the medical system 100.

FIG. 5 is a flowchart illustrating an exemplary process for determining target cardiac motion data of a subject according to some embodiments of the present disclosure. In some embodiments, process 500 may be executed by the medical system 100. For example, the process 500 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 500. The operations of the illustrated process presented below are intended to be illustrative. In some embodiments, the process 500 may be accomplished with one or more additional operations not described and/or without one or more of the operations discussed. Additionally, the order of the operations of process 500 illustrated in FIG. 5 and described below is not intended to be limiting.

In 510, the processing device 120 (e.g., the first obtaining module 410) may obtain, via a first detection device, original cardiac motion data of a subject located in an FOV of a medical device (e.g., the medical device 110).

In some embodiments, the subject (e.g., a patient) may undergo a motion (e.g., a posture motion, a physiological motion) during and/or before a scan performed by the medical device. The motion of the subject may include a posture motion and a physiological motion. As used herein, a posture motion of the subject refers to a rigid motion of a portion (e.g., the head, a leg, a hand) of the subject. For example, the rigid motion of a portion of a subject may include a translational and/or rotational motion of the portion of the subject. Exemplary rigid motion may include the rotating or nodding of the head of the subject, legs motion, hands motion, and so on. The physiological motion may include a cardiac motion, a respiratory motion, a blood flow, a gastrointestinal motion, a skeletal muscle motion, a brain motion (e.g., a brain pulsation), or the like, or any combination thereof.

The first detection device may be configured to obtain the original cardiac motion data of the subject during and/or before the scan. In some embodiments, the first detection device may include a first radar sensor. The first emission frequency of the first radar sensor may be relatively low, the wavelength of a signal emitted by the first radar sensor may be relatively long, and the penetration ability of a signal emitted by the first radar sensor may be relatively good. In some embodiments, the first radar sensor may be a Doppler radar. The Doppler radar may use the Doppler effect to detect the motion of the subject. In some embodiments, the first emission frequency of the first radar sensor may be in a range of 600 MHz˜2.4 GHz. In some embodiments, the first detection device may be configured to emit a signal (e.g., a reference signal of the first detection device as described elsewhere in the present disclosure) whose first emission frequency is such that the penetration ability of the signal is sufficient to detect a motion originated from a certain depth underneath a surface of the subject.

In some embodiments, the first radar sensor may be placed in a vicinity of the subject (e.g., placed in a vicinity of the heart of the subject) to monitor the cardiac motion of the subject. For example, the first radar sensor may be placed above the chest of the subject by a certain distance. As another example, the first radar sensor may be attached to the chest of the subject. In some embodiments, the first radar sensor may be integrated into or mounted on the medical device. For example, the first radar sensor may be mounted on an RF coil of an MRI device. As another example, the first radar sensor may be mounted on a scanning table of the medical device.

In some embodiments, the first radar sensor may obtain first detection information. For example, the first radar sensor may emit a reference signal (e.g., a beam of electromagnetic radiation waves) to an FOV of the first radar sensor, and the reference signal may be reflected by the subject. The first radar sensor may receive at least a portion of the reflected signal (e.g., an echo signal) from the subject. In some embodiments, the first detection information may include the reference signal, the received reflected signal, image data (e.g., point-cloud data) generated based on the reference signal and the received reflected signal, or the like, or any combination thereof.

The processing device 120 may determine the original cardiac motion data based on the first detection information. In some embodiments, information (e.g., a frequency difference, a phase difference) relating to the reflected signal and the reference signal may reflect a motion of the subject (e.g., a displacement, a moving velocity, a moving direction, etc., of each of one or more positions on the subject), and be used to determine the original cardiac motion data of the subject. For example, a distance between the first radar sensor and a point on the body surface of the subject (or a point inside the body of the subject) may be measured based on a frequency difference and/or a phase difference between a reference signal and a reflected signal. The variation of the distance between the first radar sensor and the point on the body surface of the subject (or the point inside the body of the subject) over a time period may be used to detect the motion of the subject.

In some embodiments, the first radar sensor mounted in a vicinity of the subject may detect the movement of the body surface of the subject, and the movement of an internal organ or tissue within the subject (e.g., the movement of the diaphragm of the heart of the subject). The movement of the body surface of the subject may be caused by a real cardiac motion and other interference motions (e.g., a respiratory motion, a posture motion) of the subject. Accordingly, the original cardiac motion data may include real cardiac motion data (i.e., target cardiac motion data as used in the present disclosure) and interference motion data (e.g., respiratory motion data, posture motion data). The real cardiac motion data may reflect the movement of one or more tissues or organs (e.g., the movement of the chest surface) caused by the cardiac motion of the subject. The interference motion data may reflect the movement of one or more tissues or organs (e.g., the movement of the chest surface) of the subject caused by one or more motions (e.g., the respiratory motion, the posture motion) of the subject other than the cardiac motion. It is understood that merely by way of example, embodiments of the present disclosure are described with reference to the cardiac motion as the motion of interest or target motion and other one or more motions as the interference motion(s). It is for illustration purposes only and not intended to be limiting. Embodiments of the present disclosure may be suitable for detection of one or more physiological motions other than the cardiac motion as the target motion(s).

In some embodiments, the original cardiac motion data may be determined by the first radar sensor, and the processing device 120 may obtain the original cardiac motion data from the first radar sensor. Alternatively, the original cardiac motion data may be determined by the first radar sensor and stored in a storage device (e.g., the storage device 130, an external source). The processing device 120 may retrieve the original cardiac motion data from the storage device. In some embodiments, the processing device 120 may obtain the original cardiac motion data from the first radar sensor in real time or intermittently (e.g., periodically or irregularly). In some embodiments, the processing device 120 may obtain the first detection information from the first radar sensor. The processing device 120 may determine the original cardiac motion data based on the first detection information.

In some embodiments, the first detection device may include an electrocardiographic device (e.g., an electrocardiograph), a pulse measuring device, or the like, that can monitor or reflect the cardiac motion of the subject. The electrocardiographic device may record an electrocardiogram (ECG) signal reflective of electric activities of a cardiac muscle. For example, electrodes may be placed on a plurality of positions (e.g., the chest, the abdomen, a shoulder) of the subject to obtain the ECG signal (i.e., the original cardiac motion data) of the subject. The pulse measuring device may be used to measure a number (or count) of pulse beats of the subject, which may reflect the cardiac motion of the subject. For example, the pulse measuring instrument may be clamped on a finger of the subject to obtain a pulse signal of the subject, and the pulse signal may be converted into a cardiac motion signal (i.e., the original cardiac motion data) of the subject.

In 520, the processing device 120 (e.g., the second obtaining module 420) may obtain, via a second detection device, detection data of the subject.

The detection data may reflect a motion state of the subject. In some embodiments, the detection data may include posture data of the subject, physiological motion data of the subject, or the like, or any combination thereof. The posture data may reflect the posture motion of the subject. In some embodiments, the posture data may include gestural information of the subject. The physiological motion data may reflect the motion of tissue or an organ that is caused or influenced by the physiological motion of the subject. In some embodiments, the detection data may include information relating to a corresponding motion of the subject. The information relating to a physiological motion may include a motion rate of each of one or more positions on the subject, a motion amplitude (or displacement) of each of one or more positions on the subject, a motion cycle, a motion phase of each of one or more positions on the subject, or the like, or any combination thereof. In some embodiments, the detection data may include a respiratory signal relating to a respiratory motion of the subject, a posture signal relating to the posture motion of the subject, or the like. For example, the respiratory signal may indicate a respiratory cycle of the subject, as well as a respiratory displacement, a respiratory rate, and/or a respiratory frequency, or the like, of each of one or more positions on the subject. The respiratory cycle may include a plurality of respiratory phases, such as an inspiratory phase (during which the chest of the subject expands and air flows into the lungs) and an expiratory phase (during which the chest shrinks and air is pushed out of the lungs).

The second detection device may be configured to obtain the detection data of the subject during and/or before the scan. In some embodiments, the second detection device may include a second radar sensor. The second emission frequency of the second radar sensor may be relatively high, the wavelength of a signal emitted by the second radar sensor may be relatively short, and the penetration ability of a signal emitted by the second radar sensor may be relatively poor. In some embodiments, the first emission frequency of the first radar sensor may be lower than a second emission frequency of the second radar sensor.

In some embodiments, the second radar sensor may include a millimeter wave radar sensor. The millimeter wave radar sensor may transmit signals with a wavelength which is in a millimeter (mm) range (e.g., 1˜10 mm). The emission frequency of the millimeter wave radar sensor may be in a range of 30˜300 GHz. A high frequency range (e.g., 30˜300 GHz) of the millimeter wave radar sensor may be used to detect a body surface movement (e.g., a skin movement) of the subject. In some embodiments, the second radar sensor may include a modulated continuous wave radar (e.g., a frequency modulated continuous wave (FMCW) radar), an unmodulated continuous-wave radar, or the like. As used herein, an FMCW radar refers to a type of radar that radiates continuous transmission power, and can change its operating frequency during the measurement; that is, the transmission signal is modulated in frequency (or in phase). Merely by way of example, the FMCW radar may include one or more transmitting antennas and one or more receiving antennas. The one or more transmitting antennas may emit a plurality of reference signals with frequencies linearly varying over time. At least a portion of the plurality of reference signals may be reflected by the surface of the subject, and a plurality of echo signals may be generated. The one or more receiving antennas may receive the plurality of echo signals. The FMCW radar may determine motion information of the subject based on information (e.g., a frequency difference, a phase difference, a time difference) relating to a reference signal and a corresponding echo signal.

In some embodiments, the second radar sensor may be mounted at one or more of various suitable locations for monitoring the motion of the subject. In some embodiments, the mounting location of the second radar sensor may be determined based on an FOV of the second radar sensor, feature information (e.g., a location, a length, a width, a thickness) of the subject, and/or the FOV of the medical device. For example, the second radar sensor may be mounted at a specific location such that the FOV of the second radar sensor can cover the entire range of the subject. As another example, the second radar sensor may be mounted at a specific location such that the FOV of the second radar sensor can cover the entire range of a scanning table. As another example, the second radar sensor may be mounted at a specific location such that the FOV of the second radar sensor can cover at least part of the FOV of the medical device. By mounting the second radar sensor at one or more of various suitable locations such that the FOV of the second radar sensor covers the entire range of the scanning table, the entire range of the subject, and/or the entire FOV of the medical device, the motion of the subject may be monitored comprehensively.

In some embodiments, the processing device 120 may determine a region of interest (ROI) of the subject. As used herein, an ROI refers to a region (e.g., an organ, tissue, a body portion) that may be significantly or primarily influenced by the respiratory motion but (substantially) not influenced by the cardiac motion of a subject such that the influence of the cardiac motion may be neglected. For example, the ROI may include the abdomen of the subject. The second radar sensor may be mounted at a specific location such that the FOV of the second radar sensor only covers the ROI of the subject. Accordingly, the respiratory motion of the subject may be (substantially) decoupled from the cardiac motion for monitoring purposes and monitored accurately and conveniently, and the respiratory motion data may be extract from the detection data easily and accurately, which may improve the accuracy of the determination of the target cardiac motion data based on the extracted respiratory motion data and the original cardiac motion data.

In some embodiments, the second radar sensor may be integrated into or mounted on the medical device. In some embodiments, the second radar sensor may be mounted outside the FOV of the medical device (e.g., on an RF coil or a main magnet of an MRI device), in order to reduce or eliminate the signal interference between the second radar sensor and the medical device (e.g., the MRI device). For example, the second radar sensor may be mounted on an upper portion of a scanning cavity (e.g., a position of the scanning cavity directly above the scanning table) of the medical device to monitor the subject on a scanning table. As another example, the second radar sensor may be mounted on a side portion of the scanning cavity of the medical device to monitor the subject on the scanning table. As still another example, a plurality of second radar sensors (e.g., a second radar sensor 610 and a second radar sensor 620 as illustrated in FIGS. 6 and 7) may be mounted on different portions of the scanning cavity (e.g., different positions of the scanning cavity above the scanning table) to monitor the subject from different directions. The number (or count) of the second radar sensors may be determined based on an FOV of the second radar sensor, the FOV of the medical device, a mounting location of the second radar sensor, and/or an installation space of the second radar sensor in the scanning cavity of the medical device. In some embodiments, each of the plurality of second radar sensors may be mounted at a specific location such that a total FOV of the plurality of second radar sensors can cover the FOV of the medical device. Merely by way of example, the plurality of second radar sensors may be mounted on different positions of the scanning cavity above the scanning table, and distributed on both sides of an axial direction (e.g., the Z-axis direction as illustrated in FIG. 1) of the medical device, so as to make a full use of the installation space of the scanning cavity, and ensure that the FOV of the plurality of second radar sensors and the FOV of the medical device may at least partially overlap, substantially coincide, or the FOV of the medical device may fall within the FOV of the plurality of second radar sensors.

In some embodiments, the second radar sensor may obtain second detection information. For example, the second radar sensor may emit a reference signal to an FOV of the second radar sensor, and the reference signal may be reflected by the subject. The second radar sensor may receive at least a portion of the reflected signal (e.g., an echo signal) from the subject. The second detection information may include the reference signal, the received reflected signal, image data (e.g., point-cloud data) generated based on the reference signal and the received reflected signal, or the like, or any combination thereof.

The processing device 120 may determine the detection data based on the second detection information. In some embodiments, information (e.g., a frequency difference, a phase difference) relating to the reflected signal and the reference signal may reflect the motion of the subject (e.g., a displacement, a moving velocity, a moving direction, etc., of each of one or more positions on the subject), and be used to determine the detection data of the subject. For example, a distance between the second radar sensor and a point on the body surface of the subject may be measured based on a frequency difference and/or a phase difference between a reference signal and a reflected signal. The variation of the distance between the second radar sensor and the point on the body surface of the subject over a time period may be used to detect the motion of the subject.

In some embodiments, the detection data may be determined by the second radar sensor, and the processing device 120 may obtain the detection data from the second radar sensor. Alternatively, the detection data may be determined by the second radar sensor and stored in a storage device (e.g., the storage device 130, or an external source). The processing device 120 may retrieve the detection data from the storage device. In some embodiments, the processing device 120 may obtain the detection data from the second radar sensor in real time or intermittently (e.g., periodically or irregularly). In some embodiments, the processing device 120 may obtain the second detection information from the second radar sensor. The processing device 120 may determine the detection data based on the second detection information.

In some embodiments, the second detection device may include an image acquisition device (e.g., a camera), a pressure sensor, an acceleration sensor, or the like, that can monitor the motion of the subject. For example, one or more cameras may be mounted around the subject to acquire images of the subject, so as to obtain the detection data of the subject. As another example, one or more acceleration sensors may be placed on the body of the subject to obtain the detection data of the subject. As still another example, a plurality of pressure sensors may be mounted on the scanning table or integrated into the scanning table. The plurality of pressure sensors may measure pressure values generated by the subject on different parts of the scanning table and obtain a pressure distribution on the scanning board. The detection data of the subject may be determined based on the pressure distribution.

In 530, the processing device 120 (e.g., the determination module 430) may determine target cardiac motion data of the subject by correcting, based on the detection data, the original cardiac motion data.

As used herein, target cardiac motion data refers to real cardiac motion data of the subject. The real cardiac motion data may indicate data regarding cardiac cycle(s) of the subject, as well as changes of the heart rate and/or cardiac motion amplitude over the cardiac cycle(s). A cardiac cycle may include a plurality of cardiac phases, such as systole (during which the left and right ventricles contract and eject blood into the aorta and pulmonary artery, respectively) and diastole (during which the ventricles are relaxed).

In some embodiments, the processing device 120 may obtain correction data by extracting at least one of the posture data or the respiratory motion data from the detection data. That is, the posture data and/or the respiratory motion data extracted from the detection data may be determined as the correction data. In some embodiments, the processing device 120 may extract the posture data from the detection data. For example, the processing device 120 may determine contour data of the subject based on the detection data. A contour of the subject may be formed by an outline of the surface of the subject. The contour data may reflect the motion of the contour of the subject. For example, the contour data may include a moving velocity (or a variation range of the moving velocity in a time period) of at least one position of a plurality of positions of the contour of the subject, a moving direction of the at least one position of the plurality of positions of the contour of the subject, a moving displacement of the at least one position of the plurality of positions of the contour of the subject, point cloud data of the contour of the subject, or the like, or any combination thereof. As used herein, point cloud data of a subject refers to a set of data points associated with the subject.

The processing device 120 may determine the posture data based on the contour data. In some embodiments, the processing device 120 may obtain a plurality of point cloud frames corresponding to a plurality of time points or a plurality of time periods acquired by the second detection device. The processing device 120 may then determine the posture data based on the plurality of point cloud frames. For example, the processing device 120 may determine the posture data by tracking a motion of at least one feature point of the subject over the plurality of time points or the plurality of time periods. The feature point may correspond to a specific physical point of the subject, such as an anatomical joint (e.g., a shoulder joint, a knee joint, an elbow joint, an ankle joint, a wrist joint) or a representative physical point in a body region (e.g., the head, the neck, a hand, a leg, a foot, a spine, a pelvis, a hip) of the subject.

In some embodiments, the processing device 120 may extract the physiological motion data of the subject from the detection data. In some embodiment, the physiological motion data may include reference cardiac motion data and the respiratory motion data. As used herein, reference cardiac motion data refers to cardiac motion data obtained from the second radar sensor. The processing device 120 may extract the reference cardiac motion data and the respiratory motion data from the detection data based on a frequency range of the respiratory motion and a frequency range of the cardiac motion according to a spectrum analysis. The frequency range of the cardiac motion and the frequency range of the cardiac motion may be manually set by a user of the medical system 100, or be determined by one or more components (e.g., the processing device 120) of the medical system 100 according to different situations. For a normal person, the frequency range of the cardiac motion may be higher than the frequency range of the cardiac motion. For example, the processing device 120 may generate filtered detection data by performing a filtering operation on the detection data to filter out a disturbed signal (e.g., the posture data). The processing device 120 may transform the filtered detection data from the time domain to the frequency domain by performing a Fourier transformation on the filtered detection data. The processing device 120 may then extract the reference cardiac motion data and the respiratory motion data from the filtered detection data in the frequency domain based on the frequency range of the respiratory motion and the frequency range of the cardiac motion. The processing device 120 may further determine the reference cardiac motion data and the respiratory motion data in the time domain by performing an inverse Fourier transformation on the reference cardiac motion data and the respiratory motion data in the frequency domain, respectively.

Further, the processing device 120 may determine the target cardiac motion data of the subject by correcting, based on the correction data, the original cardiac motion data. In some embodiments, the processing device 120 may determine the target cardiac motion data by removing the correction data (e.g., the respiratory motion data, the posture data) from the original cardiac motion data. By removing the respiratory motion data and/or the posture data from the original cardiac motion data, interference motion data caused by the respiratory motion and/or the posture motion may be removed from the original cardiac motion data, and the real cardiac motion data (i.e., the target cardiac motion data) may be obtained. In some embodiments, the original cardiac motion data and the correction data (or the detection data) may correspond to a same time point. For example, the first detection information and the second detection information may be acquired by the first detection device (e.g., the first radar sensor) and the second detection device (e.g., the second radar sensor), respectively, at the same time point. The original cardiac motion data and the correction data (or the detection data) may be determined based on the first detection information and the second detection information, respectively.

In some embodiments, the processing device 120 may obtain the target cardiac motion data by performing, based on the correction data, a filtering operation on the original cardiac motion data using an adaptive filter. In some embodiments, the processing device 120 may obtain transformed cardiac motion data by transforming the original cardiac motion data from the time domain to the frequency domain. For example, the processing device 120 may obtain the transformed cardiac motion data by performing a Fourier transformation on the original cardiac motion data. The processing device 120 may obtain transformed respiratory motion data by transforming the respiratory motion data from the time domain to the frequency domain. For example, the processing device 120 may obtain the transformed respiratory motion data by performing a Fourier transformation on the respiratory motion data. The processing device 120 may obtain candidate cardiac motion data by subtracting the transformed respiratory motion data from the transformed cardiac motion data. For example, the transformed cardiac motion data may include a first spectral component and a second spectral component. The first spectral component may correspond to the real cardiac motion data (e.g., the candidate cardiac motion data), and the second spectral component may correspond to the interference motion data caused by the respiratory motion of the subject (e.g., the transformed respiratory motion data). The first spectral component (e.g., the candidate cardiac motion data) may be obtained by subtracting the second spectral component (e.g., the transformed respiratory motion data) from the transformed cardiac motion data. Further, the processing device 120 may determine the target cardiac motion data by transforming the candidate cardiac motion data from the frequency domain to the time domain.

In 540, the processing device 120 (e.g., the determination module 430) may generate, based on the target cardiac motion data, a control signal for controlling the medical device to scan the subject.

In some embodiments, the processing device 120 may generate a control signal for controlling the medical device to scan the subject based on the target cardiac motion data and/or the respiratory motion data. In some embodiments, the control signal may involve the gating technique according to which the medical device performs the scan. Taking an MRI device as an example, the control signal may be used to cause the MRI device to start, terminate, or pause an MRI scan. In some embodiments, the processing device 120 may determine a time point (or period) in which the physiological motion of the subject is smooth or minimal based on the target cardiac motion data and/or the respiratory motion data. MR signals acquired in such a period may be minimally affected by the physiological motion and have higher signal quality compared with MR signals acquired in other periods (e.g., the systole). This may reduce physiological motion-induced artifacts in a resulting image. For example, the physiological motion at a certain time point may be regarded as being smooth or minimal if the motion amplitude at the certain time point is below a first threshold. As another example, the physiological motion in a certain period may be regarded as being smooth or minimal if, for example, a change of the motion amplitude of the physiological motion within the period is below a second threshold, or the like.

Further, the processing device 120 may cause the medical device to perform, according to the control signal, a scan on the subject. For example, the processing device 120 may determine an MR signal acquisition time based on the target cardiac motion data and/or the respiratory motion data. The MR signal acquisition time may be a time point (or period) when the MRI device is controlled to execute an MR scan on the subject. For example, the MR signal acquisition time may include a time point or period in which the physiological motion of the subject is smooth or minimal. The processing device 120 may transmit the control signal to the MRI device to execute the MR scan. By determining a suitable MR signal acquisition time based on the target cardiac motion data and/or the respiratory motion data, an image reconstructed based on MR signals detected in the MR scan may have less motion artifact and higher quality.

In some embodiments, the processing device 120 may generate an image (e.g., an MR image) of the subject based on the scan (e.g., an MR scan). The processing device 120 may perform an artifact correction on the image of the subject based on the target cardiac motion data and/or the respiratory motion data. For example, the respiratory motion data may include information regarding a respiratory signal. The processing device 120 may utilize a respiratory motion compensation technique, such as a respiratory ordered phase encoding (ROPE) technique, a centrally ordered phase encoding (COPE) technique, a hybrid ordered phase encoding (HOPE), or the like, or any combination thereof in the MR image reconstruction. For example, based on information regarding a respiratory signal, the processing device 120 may apply a same phase encoding or similar phase encodings to MR signals corresponding to a same respiratory phase or similar respiratory phases in the MR image reconstruction. In the resulting MR image, motion artifacts may be eliminated or partially eliminated.

According to some embodiments of the present disclosure, the original cardiac motion data of the subject may be obtained by the first radar sensor with a relatively low emission frequency, and the detection data of the subject may be obtained by the second radar sensor with a relatively high emission frequency. The target cardiac motion data of the subject may be determined by correcting, based on the detection data, the original cardiac motion data. In a conventional way, during a scan of a subject, one or more electrodes may be attached to the body of the subject in order to detect the physiological motion of the subject, which may cause discomfort to the subject and/or interfere with the imaging of the subject. Compared to a contact detection device (in which the detection device needs to be in physical contact with a subject for detecting motion data of the subject), the non-contact detection device (e.g., the first radar sensor, the second radar sensor) disclosed herein may reduce the discomfort of the subject, reduce interference of the imaging by a contact detection device attached on the subject, and/or avoid the procedure and time needed for setting up such a contact detection device on the subject, which in turn may reduce the setup time of the medical device. Moreover, a control signal for controlling the medical device to scan the subject may be generated based on the target motion data (e.g., target cardiac motion data), which may reduce physiological motion-induced artifacts in a resulting image.

In some embodiments, before operation 540, the processing device 120 may determine whether the posture data (or the gestural information) during an operation, e.g., a scan of the subject, is in a threshold range. The threshold range may be determined manually by a user (e.g., a doctor) of the medical system 100 or by one or more components (e.g., the processing device 120) of the medical system 100 according to different situations. For example, the processing device 120 may determine whether a moving amplitude of at least one point on the contour of the subject is greater than an amplitude threshold. In response to determining that a moving amplitude of a point on the contour of the subject is greater than the amplitude threshold, the processing device 120 may determine that the posture data exceeds the threshold range. As another example, the processing device 120 may determine whether a moving velocity of at least one point on the contour of the subject is greater than a velocity threshold. In response to determining that a moving velocity of a point on the contour of the subject is greater than the velocity threshold, the processing device 120 may determine that the posture data exceeds the threshold range.

In response to determining that the posture data exceeds the threshold range, it may indicate that the impact of the posture motion of the subject on the scan data acquired by the medical device is non-negligible, and an image reconstructed based on the scan data may include non-negligible motion-induced artifacts. In some embodiments, in response to determining that the posture data exceeds the threshold range, the processing device 120 may cause the medical device to terminate or pause the operation. In some embodiments, in response to determining that the posture data exceeds the threshold range, the processing device 120 may mark the scan data obtained during the posture motion of the subject, and the marked scan data may be discarded and not used for image reconstruction. Thus, the quality of the operation, e.g., assessed based on the quality of an image generated based on the scan, may be improved and the operation time may be saved.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 120 may extract data associated with other physiological motions (e.g., a blood flow, a gastrointestinal motion, a skeletal muscle motion, a brain motion) from the detection data of the subject acquired by the second detection device. The processing device 120 may determine the target cardiac motion data of the subject by correcting, based on the data associated with other physiological motions, the original cardiac motion data.

FIGS. 6 and 7 are schematic diagrams illustrating an exemplary medical system according to some embodiments of the present disclosure.

As illustrated in FIGS. 6 and 7, a medical system 600 may include a first radar sensor and two second radar sensors (e.g., a second radar sensor 610, a second radar sensor 620). The first radar sensor may acquire original cardiac motion data of a subject 601 located in an FOV 670 of a medical device. The first radar sensor may include an antenna 630, a cable 640, and a signal processor 650. The antenna 630 may be placed on the chest of the subject 601. The antenna 630 may transmit reference signals to an FOV to the first radar sensor, and receive echo signals reflected by the subject 601. The cable 640 may connect the antenna 630 and the signal processor 650 to implement data transmission between the antenna 630 and the signal processor 650. The signal processor 650 may be placed under a scanning table of the medical device. The signal processor 650 may determine the original cardiac motion data of the subject 601 based on information (e.g., a frequency difference, a phase difference) relating to the echo signals and the reference signals.

The second radar sensor 610 and the second radar sensor 620 may acquire detection data (e.g., posture data, physiological motion data) of the subject 601. The second radar sensor 610 and the second radar sensor 620 may be mounted on an upper portion of a scanning cavity of the medical device. In some embodiments, the second radar sensor 610 and the second radar sensor 620 may be mounted outside the FOV 670 of the medical device. Accordingly, the signal interference between the second radar sensors (e.g., the second radar sensor 610, the second radar sensor 620) and the medical device (e.g., an MRI device) may be reduced or eliminated by mounting the second radar sensors (e.g., the second radar sensor 610, the second radar sensor 620) outside the FOV 670 of the medical device.

In some embodiments, as illustrated in FIG. 6, the second radar sensor 610 and the second radar sensor 620 may be mounted on two sides of the FOV 670 of the medical device along an axial direction (e.g., the Z-axis direction as illustrated in FIG. 1) of a scanning cavity of the medical device. The second radar sensor 610 and a surface of the scanning cavity of the medical device may form a first angle A. The second radar sensor 620 and the surface of the scanning cavity of the medical device may form a second angle B. A first FOV of the second radar sensor 610 and a second FOV of the second radar sensor 620 may cover the FOV 670 of the medical device.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the medical system 600 may include two or more first radar sensors and/or three or more second radar sensors.

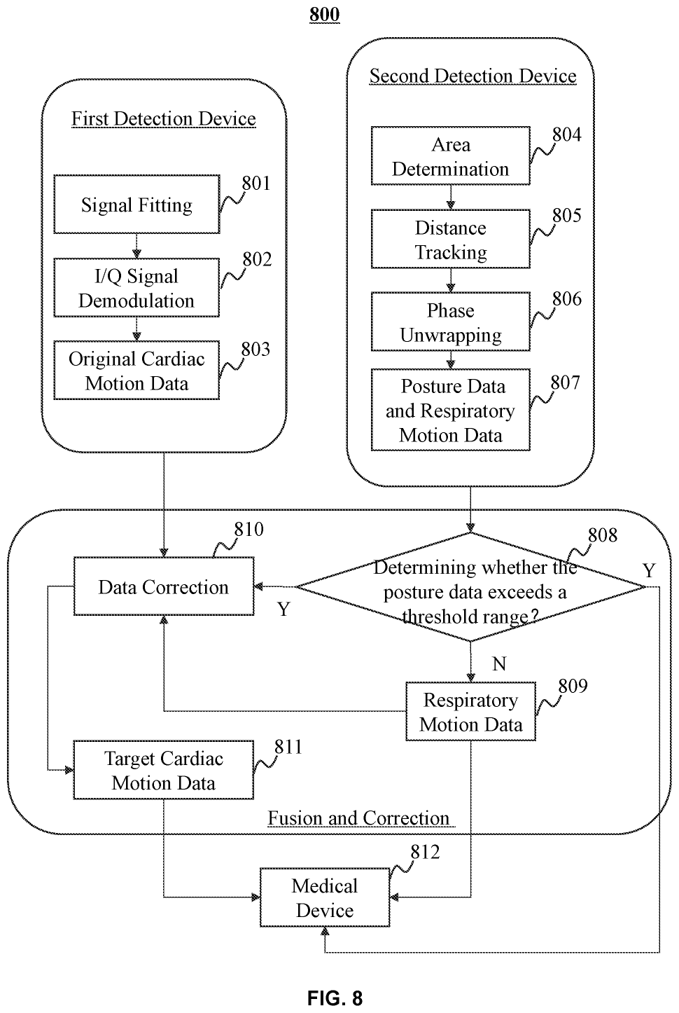

FIG. 8 is a flowchart illustrating an exemplary process for determining target cardiac motion data of a subject according to some embodiments of the present disclosure. In some embodiments, process 800 may be executed by the medical system 100. For example, the process 800 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 800. The operations of the illustrated process presented below are intended to be illustrative. In some embodiments, the process 800 may be accomplished with one or more additional operations not described and/or without one or more of the operations discussed. Additionally, the order of the operations of process 800 illustrated in FIG. 8 and described below is not intended to be limiting.

In 801, the processing device 120 (e.g., the first obtaining module 410) or a first radar sensor (e.g., the first radar sensor as described in FIG. 5) may perform a signal fitting operation on first detection information obtained by the first radar sensor.

In some embodiments, the first radar sensor may obtain the first detection information by monitoring a motion of a body region (e.g., a chest region) near the heart of the subject. In some embodiments, the processing device 120 may perform a circle fitting operation on the first detection information according to a least square algorithm to generate a fitted first detection information.

In 802, the processing device 120 (e.g., the first obtaining module 410) or the first radar sensor may perform an I/O signal demodulation operation on the fitted first detection information.

In some embodiments, the processing device 120 may perform an arctangent operation on the fitted first detection information to obtain a demodulated signal. The demodulated signal may include a phase difference relating to echo signals and reference signals of the first radar sensor caused by a real cardiac motion and an interference motion (e.g., a respiratory motion, a posture motion) of the subject.

In 803, the processing device 120 (e.g., the first obtaining module 410) or the first radar sensor may obtain original cardiac motion data of the subject based on the demodulated signal.

In some embodiments, the processing device 120 may perform a Fourier transformation on the demodulated signal to obtain the original cardiac motion data. The original cardiac motion data may include real cardiac motion data (i.e., target cardiac motion data) and interference motion data (e.g., respiratory motion data, posture motion data).

In 804, the processing device 120 (e.g., the second obtaining module 420) or a second radar sensor (e.g., the second radar sensor as described in FIG. 5) may determine an ROI of the subject, and monitor a motion of the ROI of the subject.

In some embodiments, the ROI may be a region (e.g., an organ, tissue, a body portion) that may be significantly influenced by the physiological motion of the subject. For example, the ROI may include the chest, the abdomen, the neck, or the like, of the subject. In some embodiments, the ROI may be a region (e.g., an organ, tissue, a body portion) that may be significantly or primarily influenced by the respiratory motion but (substantially) not influenced by the cardiac motion of a subject such that the influence of the cardiac motion may be neglected. For example, the ROI may include the abdomen of the subject.

In 805, the processing device 120 (e.g., the second obtaining module 420) or the second radar sensor may track a distance between the ROI and the second radar sensor.

In some embodiments, the distance between the ROI and the second radar sensor may be tracked based on second detection information obtained by the second radar sensor. The second detection information may be obtained by the second radar sensor by monitoring the motion of the ROI of the subject. For example, motion information (e.g., a moving displacement, a moving velocity, a moving direction) of at least one position of the ROI of the subject may be determined based on information (e.g., a frequency difference, a phase difference) relating to echo signals and reference signals of the second radar sensor.

In 806, the processing device 120 (e.g., the second obtaining module 420) or the second radar sensor may obtain detection data of the subject.

The detection data may include posture data and/or physiological motion data of the subject. In some embodiments, the processing device 120 may perform a phase unwrapping operation on the detection data. As used herein, a phase unwrapping operation refers to a process of exacting a plurality of sets of motion data related to a plurality of motions from the detection data based on motion frequency ranges of the plurality of motions. For example, the processing device 120 transform the detection data from the time domain to the frequency domain by performing a Fourier transformation on the detection data. The processing device 120 may extract reference cardiac motion data and the respiratory motion data from the detection data in the frequency domain based on the frequency range of the respiratory motion and the frequency range of the cardiac motion.

In 807, the processing device 120 (e.g., the second obtaining module 420) or the second radar sensor may obtain respiratory motion data and posture data.

For example, the processing device 120 may determine the respiratory motion data in the time domain by performing an inverse Fourier transformation on the respiratory motion data in the frequency domain. As another example, the processing device 120 may determine contour data of the subject based on the detection data. The processing device 120 may determine the posture data based on the contour data.

In 808, the processing device 120 (e.g., the determination module 430) may determine whether the posture data exceeds a threshold range.

For example, the processing device 120 may determine whether a moving amplitude of at least one point on the contour of the subject is greater than an amplitude threshold. In response to determining that a moving amplitude of a point on the contour of the subject is greater than the amplitude threshold, the processing device 120 may determine that the posture data exceeds the threshold range. As another example, the processing device 120 may determine whether a moving velocity of at least one point on the contour of the subject is greater than a velocity threshold. In response to determining that a moving velocity of a point on the contour of the subject is greater than the velocity threshold, the processing device 120 may determine that the posture data exceeds the threshold range.

In response to determining that the posture data does not exceed the threshold range, process 800 may proceed to operation 809. In 809, the processing device 120 (e.g., the obtaining module 1010) may correct the original cardiac motion data based on the respiratory motion data.

In some embodiments, in response to determining that the posture data exceeds the threshold range, process 800 may proceed to operation 810. In some embodiments, in response to determining that the posture data exceeds the threshold range, process 800 may proceed to operation 812.

In 810, the processing device 120 (e.g., the determination module 430) may correct the original cardiac motion data based on the respiratory motion data and/or the posture data.

In 811, the processing device 120 (e.g., the determination module 430) may determine target cardiac motion data of the subject.

In some embodiments, the processing device 120 may obtain transformed cardiac motion data by transforming the original cardiac motion data from the time domain to the frequency domain. The processing device 120 may obtain transformed respiratory motion data by transforming the respiratory motion data from the time domain to the frequency domain. The processing device 120 may obtain candidate cardiac motion data by subtracting the transformed respiratory motion data from the transformed cardiac motion data. Further, the processing device 120 may determine the target cardiac motion data by transforming the candidate cardiac motion data from the frequency domain to the time domain. Operations 810 and 811 may be performed in a similar manner as operation 530 as described in connection with FIG. 5, the descriptions of which are not repeated here.

In 812, the processing device 120 (e.g., the determination module 430) may generate a control signal for controlling the medical device to scan the subject based on the target cardiac motion data, the respiratory motion data, and/or the posture data.

For example, in response to determining that the posture data exceeds the threshold range, the processing device 120 may cause the medical device to terminate or pause the scan. As another example, the processing device 120 may determine a signal acquisition time (e.g., an MR signal acquisition time) based on the target cardiac motion data, the respiratory motion data, and/or the posture data according to a gating technique, as described in connection with operation 540. The medical device may scan the subject based on the signal acquisition time.

According to some embodiments of the present disclosure, the detection data of the subject obtained by the second radar sensor (e.g., a millimeter wave radar sensor) mounted on an upper portion of a scanning cavity of the medical device may be used to correct the original cardiac motion data of the subject obtained by the first radar sensor (e.g., a Doppler radar) placed in a vicinity of the heart of the subject. The Doppler radar cannot identify distance information due to the working principle of Doppler radar, and the original cardiac motion data may include real cardiac motion data (i.e., target cardiac motion data as used in the present disclosure) and interference motion data (e.g., respiratory motion data). The interference motion data may be removed from the original cardiac motion data based on the detection data obtained by the second radar sensor, as described in connection with operation 530. The control signal for controlling the medical device to scan the subject may be generated based on the target cardiac motion data, which may reduce physiological motion-induced artifacts in a resulting image. In addition, the posture motion data may be obtained by the second radar sensor. The processing device 120 may determine whether the posture data during an operation, e.g., a scan of the subject, is in a threshold range, as described in connection with operation 540. In some embodiments, in response to determining that the posture data exceeds the threshold range, the processing device 120 may cause the medical device to terminate or pause the operation. Thus, the quality of the operation, e.g., assessed based on the quality of an image generated based on the scan, may be improved and the operation time may be saved.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

What is claimed is:

1. A method for motion detection, which is implemented on a computing device including at least one processor and at least one storage device, comprising:
obtaining, via a first detection device, original cardiac motion data of a subject located in a field of view (FOV) of a medical device;
obtaining, via a second detection device, detection data of the subject, wherein the detection data includes at least one of posture data of the subject or physiological motion data of the subject; and
determining target cardiac motion data of the subject by correcting, based on the detection data, the original cardiac motion data.

2. The method of claim 1, further comprising:
generating, based on the target cardiac motion data, a control signal for controlling the medical device to scan the subject; and
causing the medical device to perform, according to the control signal, a scan on the subject.

3. The method of claim 1, wherein the physiological motion data includes respiratory motion data, and the determining target cardiac motion data of the subject by correcting, based on the detection data, the original cardiac motion data comprises:
obtaining correction data by extracting at least one of the posture data or the respiratory motion data from the detection data; and
determining the target cardiac motion data of the subject by correcting, based on the correction data, the original cardiac motion data.

4. The method of claim 3, wherein the determining the target cardiac motion data of the subject by correcting, based on the correction data, the original cardiac motion data comprises:
determining the target cardiac motion data of the subject by removing the correction data from the original cardiac motion data.

5. The method of claim 4, wherein the correction data includes respiratory motion data, and the determining the target cardiac motion data of the subject by removing the correction data from the original cardiac motion data comprises:
obtaining transformed cardiac motion data by transforming the original cardiac motion data from a time domain to a frequency domain;
obtaining transformed respiratory motion data by transforming the respiratory motion data from the time domain to the frequency domain;
obtaining candidate cardiac motion data by subtracting the transformed respiratory motion data from the transformed cardiac motion data; and
determining the target cardiac motion data by transforming the candidate cardiac motion from the frequency domain to the time domain.

6. The method of claim 2, wherein the control signal involves a gating technique according to which the medical device performs the scan.

7. The method of claim 1, wherein the first detection device includes at least one of a first radar sensor, an electrocardiographic device, or a pulse measuring device.

8. The method of claim 7, wherein the second detection device includes at least one of a second radar sensor, an image acquisition device, a pressure sensor, or an acceleration sensor.

9. The method of claim 8, wherein a first emission frequency of the first radar sensor is lower than a second emission frequency of the second radar sensor.

10. The method of claim 9, wherein
the first radar sensor is a Doppler radar,
the first emission frequency is in a range of 600 MHz˜2.4 GHz, and
the second radar sensor is a millimeter wave radar sensor.

11. The method of claim 10, wherein a plurality of second radar sensors are mounted on different portions of a scanning cavity of the medical device to monitor the subject from different directions.

12. The method of claim 1, wherein the medical device includes at least one of a magnetic resonance imaging (MRI) device, a positron emission tomography (PET) device, a single-photon emission computed tomography (SPECT) device, a computed tomography (CT) device, or an X-ray imaging device.

13. A system for motion detection, comprising:
at least one storage device including a set of instructions; and
at least one processor configured to communicate with the at least one storage device, wherein when executing the set of instructions, the at least one processor is configured to direct the system to perform operations including:
obtaining, via a first detection device, original cardiac motion data of a subject located in a field of view (FOV) of a medical device;
obtaining, via a second detection device, detection data of the subject, wherein the detection data includes at least one of posture data of the subject or physiological motion data of the subject; and
determining target cardiac motion data of the subject by correcting, based on the detection data, the original cardiac motion data.

14. The system of claim 13, wherein the at least one processor is configured to direct the system to perform operations including:
generating, based on the target cardiac motion data, a control signal for controlling the medical device to scan the subject; and
causing the medical device to perform, according to the control signal, a scan on the subject.

15. The system of claim 13, wherein the physiological motion data includes respiratory motion data, and the determining target cardiac motion data of the subject by correcting, based on the detection data, the original cardiac motion data comprises:
obtaining correction data by extracting at least one of the posture data or the respiratory motion data from the detection data; and
determining the target cardiac motion data of the subject by correcting, based on the correction data, the original cardiac motion data.

16. The system of claim 15, wherein the determining the target cardiac motion data of the subject by correcting, based on the correction data, the original cardiac motion data comprises:
determining the target cardiac motion data of the subject by removing the correction data from the original cardiac motion data.

17. The system of claim 16, wherein the correction data includes respiratory motion data, and the determining the target cardiac motion data of the subject by removing the correction data from the original cardiac motion data comprises:
obtaining transformed cardiac motion data by transforming the original cardiac motion data from a time domain to a frequency domain;
obtaining transformed respiratory motion data by transforming the respiratory motion data from the time domain to the frequency domain;
obtaining candidate cardiac motion data by subtracting the transformed respiratory motion data from the transformed cardiac motion data; and
determining the target cardiac motion data by transforming the candidate cardiac motion from the frequency domain to the time domain.

18. The system of claim 14, wherein the control signal involves a gating technique according to which the medical device performs the scan.

19. The system of claim 13, wherein the first detection device includes at least one of a first radar sensor, an electrocardiographic device, or a pulse measuring device.

20. A non-transitory computer readable medium, comprising executable instructions that, when executed by at least one processor, direct the at least one processor to perform a method for motion correction, the method comprising:
obtaining, via a first detection device, original cardiac motion data of a subject located in a field of view (FOV) of a medical device;
obtaining, via a second detection device, detection data of the subject, wherein the detection data includes at least one of posture data of the subject or physiological motion data of the subject; and
determining target cardiac motion data of the subject by correcting, based on the detection data, the original cardiac motion data.
